(19) United States
(12) Patent Application Publication
(10) Pub. No.: US 2017/0233628 A1
(43) Pub. Date: Aug. 17, 2017

(54) ENZYME DESTABILIZERS FOR DESTABILIZING ENZYMES PRODUCING SULFUR CONTAINING COMPOUNDS IN DOWNHOLE FLUIDS

(71) Applicant: BAKER HUGHES INCORPORATED, Houston, TX (US)

(72) Inventor: Charles David Armstrong, Tomball, TX (US)

(73) Assignee: BAKER HUGHES INCORPORATED, Houston, TX (US)

(21) Appl. No.: 15/431,200

(22) Filed: Feb. 13, 2017

Related U.S. Application Data
(60) Provisional application No. 62/295,303, filed on Feb. 15, 2016.

(51) Int. Cl. C09K 8/035; C12N 9/12; C12N 9/02; C12N 9/10; C09K 8/62; C09K 8/582

(52) U.S. Cl. CPC C09K 8/035; C09K 8/62; C09K 8/582; C12Y 207/07004; C12Y 108/99002; C12Y 108/99001; C12Y 108/03001; C12Y 108/99003; C12Y 205/01065; C12Y 205/01047; C12N 9/0051; C12N 9/1085; C12N 9/1241; C09K 2208/20

(57) ABSTRACT

Methods and fluid compositions are provided for decreasing an amount of sulfur-containing compounds in downhole fluids and/or subterranean reservoir wellbores by including at least one enzyme destabilizer in a fluid composition. The fluid composition may then be circulated into a subterranean reservoir wellbore. The fluid composition may further include a base fluid and at least one sulfur producing enzyme. The base fluid may be or include, but is not limited to, drilling fluids, servicing fluids, production fluids, completion fluids, injection fluids, refinery fluids, and combinations thereof. The enzyme destabilizer(s) may be destabilize the sulfur producing enzymes and thereby decrease an amount of sulfur-containing compounds produced vis-à-vis the sulfur producing enzyme(s).

### CROSS-REFERENCE TO RELATED APPLICATION

This application claims the benefit of Provisional Patent Application No. 62/295,303 filed Feb. 15, 2016, which is incorporated by reference herein in its entirety.

### TECHNICAL FIELD

The present invention relates to fluid compositions and methods for decreasing or removing sulfur-containing compounds by decreasing the ability of sulfur producing enzymes to produce the sulfur containing compounds.

### BACKGROUND

The presence of sulfur species in hydrocarbon fluids and aqueous streams is undesirable for various reasons. The subterranean reservoirs currently being developed have increased amounts of sulfur species within the produced hydrocarbon streams (oil and gas). Hydrogen sulfide is a toxic gas that is heavier than air and is very corrosive to well and surface equipment.

During combustion, sulfur-rich hydrocarbon streams also produce heavy environmental pollution. When sulfur-rich streams contact metals, sulfur species lead to brittleness in carbon steels and to stress corrosion cracking in more highly alloyed materials. Moreover, hydrogen sulfides in various hydrocarbon or aqueous streams pose a safety hazard and a corrosion hazard. A quick removal of these odorous and environmentally malicious species would be desirable in both oilfield and refinery operations.

For the reasons mentioned, attempts have been made to wash out, or chemically convert, the sulfur species from hydrocarbon fluids and aqueous systems. Several classes of chemicals, also known as sweeteners, are available for removing sulfur species from a hydrocarbon or aqueous stream, but many of them have serious limitations. For example, nitrogen-containing hydrogen sulfide sweeteners, such as hydrotriazine-based additives, have been in the industry for a long while now. However, the amines released while scavenging the sulfur species pose an overhead corrosion threat in various downstream processes, including distillation columns. Formaldehyde is a nitrogen-free sweetener, but it is also a potential carcinogen. Glyoxal is another nitrogen-free hydrogen sulfide sweetener, but its application is often limited due to its corrosivity and low boiling point. Metal oxides have also been proposed, but such applications are narrowed by the handling challenges and solid residual formation concerns to downstream refining catalysts and processes. Acrolein is a clean and extremely potent hydrogen sulfide/mercaptan sweetener, but it requires special handling due to toxicity concerns.

Sulfur-containing compounds are deleterious in the subterranean reservoir wellbores in which they reside. Additives may be added to the downhole fluids for circulation into the reservoir wellbore. The downhole fluids may be or include drilling fluids, completion fluids, servicing fluids (e.g. fracturing fluids), production fluids, injection fluids, and combinations thereof. Drilling fluids are typically classified according to their base fluid. In water-based fluids, solid particles, such as weighting agents, are suspended in a continuous phase consisting of water or brine. Oil can be emulsified in the water, which is the continuous phase. “Water-based fluid” is used herein to include fluids having an aqueous continuous phase where the aqueous continuous phase can be all water or brine, an oil-in-water emulsion, or an oil-in-brine emulsion. Brine-based fluids, of course are water-based fluids, in which the aqueous component is brine.

Oil-based fluids are the opposite or inverse of water-based fluids. “Oil-based fluid” is used herein to include fluids having a non-aqueous continuous phase where the non-aqueous continuous phase is all oil, a non-aqueous fluid, a water-in-oil emulsion, a water-in-non-aqueous emulsion, a brine-in-oil emulsion, or a brine-in-non-aqueous emulsion. In oil-based fluids, solid particles are suspended in a continuous phase consisting of oil or another non-aqueous fluid. Water or brine can be emulsified in the oil; therefore, the oil is the continuous phase. In oil-based fluids, the oil may consist of any oil or water-immiscible fluid that may include, but is not limited to, diesel, mineral oil, esters, refinery cuts and blends, or alpha-olefins. Oil-based fluid as defined herein may also include synthetic-based fluids or muds (SBMs), which are synthetically produced rather than refined from naturally-occurring materials. Synthetic-based fluids often include, but are not necessarily limited to, olefin oligomers of ethylene, esters made from vegetable fatty acids and alcohols, ethers and polyethers made from alcohols and polyalcohols, paraffinic, or aromatic, hydrocarbons alkyl benzenes, terpenes and other natural products and mixtures of these types.

There are a variety of functions and characteristics that are expected of completion fluids. The completion fluid may be placed in a well to facilitate final operations prior to initiation of production. Completion fluids are typically brines, such as chlorides, bromides, and/or formates, but may be any non-damaging fluid having proper density and flow characteristics. Suitable salts for forming the brines include, but are not necessarily limited to, sodium chloride, calcium chloride, zinc chloride, potassium chloride, potassium bromide, sodium bromide, calcium bromide, zinc bromide, sodium formate, potassium formate, ammonium formate, cesium formate, and mixtures thereof. Chemical compatibility of the completion fluid with the reservoir formation and formation fluids is key. Chemical additives, such as polymers and surfactants are known in the art for being introduced to the brines used in well servicing fluids for various reasons that include, but are not limited to, increasing viscosity, and increasing the density of the brine. Completion fluids do not contain suspended solids.

Production fluid is the fluid that flows from a formation to the surface of an oil well. These fluids may include oil, gas, water, as well as any contaminants (e.g. H₂S, asphaltenes, etc.). The consistency and composition of the production fluid may vary.

Refinery fluids are fluids that may be further processed or refined at a refinery. A non-limiting example of a refinery process may include reducing or preventing the formation of foulants. Non-limiting examples of foulants may be or include hydrates, asphaltenes, coke, coke precursors, naphthenates, inorganic solid particles (e.g. sulfates, oxides, scale, and the like), and combinations thereof. Non-limiting examples of fluids to be refined include crude oil, production water, and combinations thereof.

Servicing fluids, such as remediation fluids, stimulation fluids, workover fluids, and the like, have several functions and characteristics necessary for repairing a damaged well. Such fluids may be used for breaking emulsions already formed and for removing formation damage that may have occurred during the drilling, completion and/or production operations. The terms “remedial operations” and “remediate” are defined herein to include a lowering of the viscosity of gel damage and/or the partial or complete removal of damage of any type from a subterranean formation. Similarly, the term “remediation fluid” is defined herein to include any fluid that may be useful in remedial operations. A stimulation fluid may be a treatment fluid prepared to stimulate, restore, or enhance the productivity of a well, such as fracturing fluids and/or matrix stimulation fluids in one non-limiting example.

Hydraulic fracturing is a type of stimulation operation, which uses pump rate and hydraulic pressure to fracture or crack a subterranean formation in a process for improving the recovery of hydrocarbons from the formation. Once the crack or cracks are made, high permeability proppant relative to the formation permeability is pumped into the fracture to prop open the crack. When the applied pump rates and pressures are reduced or removed from the formation, the crack or fracture cannot close or heal completely because the high permeability proppant keeps the crack open. The propped crack or fracture provides a high permeability path connecting the producing wellbore to a larger formation area to enhance the production of hydrocarbons.

The development of suitable fracturing fluids is a complex art because the fluids must simultaneously meet a number of conditions. For example, they must be stable at high temperatures and/or high pump rates and shear rates that can cause the fluids to degrade and prematurely settle out the proppant before the fracturing operation is complete. Various fluids have been developed, but most commercially used fracturing fluids are aqueous based liquids that have either been gelled or foamed to better suspend the proppants within the fluid.

Injection fluids may be used in enhanced oil recovery (EOR) operations, which are sophisticated procedures that use viscous forces and/or interfacial forces to increase the hydrocarbon production, e.g. crude oil, from oil reservoirs. The EOR procedures may be initiated at any time after the primary productive life of an oil reservoir when the oil production begins to decline. The efficiency of EOR operations may depend on reservoir temperature, pressure, depth, net pay, permeability, residual oil and water saturations, porosity, fluid properties, such as oil API gravity and viscosity, and the like.

EOR operations are considered a secondary or tertiary method of hydrocarbon recovery and may be necessary when the primary and/or secondary recovery operation has left behind a substantial quantity of hydrocarbons in the subterranean formation. Primary methods of oil recovery use the natural energy of the reservoir to produce oil or gas and do not require external fluids or heat as a driving energy; EOR methods are used to inject materials into the reservoir that are not normally present in the reservoir.

Secondary EOR methods of oil recovery inject external fluids into the reservoir, such as water and/or gas, to re-pressurize the reservoir and increase the oil displacement. Tertiary EOR methods include the injection of special fluids, such as chemicals, miscible gases and/or thermal energy. The EOR operations follow the primary operations and target the interplay of capillary and viscous forces within the reservoir. For example, in EOR operations, the energy for producing the remaining hydrocarbons from the subterranean formation may be supplied by the injection of fluids into the formation under pressure through one or more injection wells penetrating the formation, whereby the injection fluids drive the hydrocarbons to one or more producing wells penetrating the formation. EOR operations are typically performed by injecting the fluid through the injection well into the subterranean reservoir to restore formation pressure, improve oil displacement or fluid flow in the reservoir, and the like.

Examples of EOR operations include water-based flooding and gas injection methods. Water-based flooding may also be termed ‘chemical flooding’ if chemicals are added to the water-based injection fluid. Water-based flooding may be or include, polymer flooding, ASP (alkali/surfactant/polymer) flooding, SP (surfactant/polymer) flooding, low salinity water and microbial EOR; gas injection includes immiscible and miscible gas methods, such as carbon dioxide flooding, and the like.

It would be desirable if additives were developed for fluid compositions used during hydrocarbon recovery to decrease or remove sulfur-containing compounds in recovered downhole fluids and in hydrocarbon reservoir wellbores.

### SUMMARY

There is provided, in one form, a fluid composition including a base fluid, at least one sulfur producing enzyme, and at least one enzyme destabilizer. The fluid composition may include a decreased amount of sulfur-containing compounds in the presence of the enzyme destabilizer(s) as compared to an otherwise identical fluid absent the enzyme destabilizer(s). The base fluid may be or include, but is not limited to, drilling fluids, servicing fluids, production fluids, completion fluids, injection fluids, refinery fluids, and combinations thereof. The enzyme destabilizer(s) may be or include, but is not limited to a denaturant, an active site inhibitor, an allosteric inhibitor, and combinations thereof.

There is provided, in another non-limiting form, a method including circulating a fluid composition into a subterranean reservoir wellbore, and decreasing an amount of the sulfur-containing compounds derived from at least one sulfur producing enzyme in the subterranean reservoir wellbore and/or downhole fluids recovered therefrom as compared to an identical fluid composition absent the enzyme destabilizers. The fluid composition may include at least one enzyme destabilizer in an effective concentration to decrease an amount of sulfur-containing compounds in the fluid composition. The enzyme destabilizer(s) may be or include, but are not limited to, a denaturant, an active site inhibitor, an allosteric inhibitor, and combinations thereof. ‘Derived from’ with respect to the sulfur producing enzyme is meant to include any product naturally produced from a reaction involving the sulfur producing enzyme(s).

The enzyme destabilizer(s) destabilize sulfur producing enzyme(s) of a sulfur producing bacteria and thereby prevent and/or decrease an amount of sulfur-containing compounds produced therefrom.

### BRIEF DESCRIPTION OF THE DRAWINGS

FIG. 1 illustrates the ability of sterile solutions to produce hydrogen sulfide (H₂S); and

FIG. 2 indicates the treatment of samples comprising sulfur producing enzymes after treatment with inhibitors.

### DETAILED DESCRIPTION

It has been discovered that at least one enzyme destabilizer may be used to destabilize at least one sulfur producing enzyme in downhole fluids to decrease or remove sulfur-containing compounds in the downhole fluids as compared to an otherwise identical fluid absent the enzyme destabilizer(s). The downhole fluid may be or include, but is not limited to drilling fluids, servicing fluids, production fluids, completion fluids, injection fluids, refinery fluids, and combinations thereof. Enzyme destabilizer(s) may decrease sulfur-containing compounds, e.g. hydrogen sulfide (H₂S) in a non-limiting embodiment, within a subterranean reservoir wellbore and any fluids recovered therefrom.

In another non-limiting embodiment, the fluid composition or a base fluid incorporated thereinto may be contained in an oil pipeline, gas pipeline, a refinery (e.g. separation vessels, dehydration units, gas lines, and pipelines), and combinations thereof. In yet another non-limiting embodiment, the fluid composition may include a salt, such as but not limited to, a brine, sea salt, and combinations thereof. The brine may be or include, but is not limited to potassium chloride, sodium chloride, calcium chloride, zinc chloride, potassium bromide, sodium bromide, calcium bromide, zinc bromide, sodium formate, potassium formate, ammonium formate, cesium formate, and combinations thereof.

“Enzyme destabilizer” is defined herein to include any substance that would decrease or inhibit a sulfur producing enzyme's ability from producing a sulfur-containing compound. ‘Sulfur producing enzyme’ is defined herein to be an enzyme that produces at least one sulfur containing compound from a reaction involving the sulfur producing enzyme. For example, the sulfur producing enzyme may directly produce the sulfur containing compound, itself, or the enzyme may indirectly aid in the production of the sulfur containing compound. In another non-limiting embodiment, the sulfur producing enzymes are prevented or inhibited from metabolizing sulfur containing compounds into hydrogen sulfide.

The enzyme destabilizer(s) may be or include a denaturant, an active site inhibitor, an allosteric inhibitor, and combinations thereof. Non-limiting examples of the enzyme destabilizer(s) may be or include, but not limited to, malimide, sodium flurophosphate, iodoacetamide, sodium dodecyl sulfate, sodium chromium tetrahydrate, 5,5′-dithiobis(2-nitrobenzoic acid), sodium molybdate, and combinations thereof.

Denaturants may bind to the sulfur producing enzyme and decrease the hydrophobic and electrostatic driving forces required for proper enzyme folding and three dimensional structure of the sulfur producing enzyme. Treatment of the sulfur producing enzyme with a denaturant may result in inactivation of the sulfur producing enzyme. Non-limiting examples of denaturants may be or include sodium dodecyl sulfate, guanidine HCl, Acetic Acid, Trichloroacetic Acid, Sulfosalicylic Acid, Sodium bicarbonate, Ethanol, Methanol, formaldehyde, glutaraldehyde, Urea, Lithium perchlorate, 2-Mercaptoethanol, Dithiothreitol, tris(2-carboxyethyl)phosphine, picric acid, acids and bases capable of altering the pH stability of the protein, chelators (e.g. EDTA and 1,10 Phenanthroline), that remove metal centers from the active site, and combinations thereof.

Active site inhibitors may covalently bind in an active site of the sulfur producing enzymes and thereby permanently disable the sulfur producing enzymes from binding the substrate for producing sulfur containing compounds. Thus, an active site inhibitor prevents the sulfur producing enzyme from producing sulfur-containing compounds. Non-limiting examples of active site inhibitors useful for disabling the sulfur producing enzymes from sulfur containing compounds may be or include, but not limited to, alkalating agents, iodine containing compounds (e.g. iodoacetamide), MoO₄²⁻. FPO₃²⁻, FSO³⁻, SeO₄²⁻, CrO₄²⁻, 3′ phosphoadenosine 5′ phosphosulfate, Adenoside 5′phosphoamidate, Adenosine-monosulfate, Adenoside 5′ phosphosulfate, 5′,5-dithiobis(2 nitrobenzoic acid), 8-hydroxyquinoline, diethyldithiocarbomate, pyridoxal phosphate, pyridoxine, EDTA, 1,10 phenanthroline, iodonium diphenyl chloride, N-ethylmaleimide, Adenosine monophosphate, adenosine diphosphate, adenosine triphosphate, phenylglyoxal, Mg²⁺, S₂O₃²⁻, diethyldithiocarbomate, idonium diphenyl chloride, and combinations thereof.

An allosteric inhibitor may bind to another area of the sulfur producing enzyme; ‘another area’ is defined to be any other area of the sulfur producing enzyme that is not the active site of the sulfur producing enzyme. By binding another area of the enzyme, the geometry of the active site of the sulfur producing enzyme may change. The change to the active site disables the sulfur producing enzyme from accepting or accommodating its conventional substrate. Non-limiting examples of the allosteric inhibitors may be or include, but not limited to, 3′-phosphoadenosine-5′-phosphosulfate (PAPS), monovalent cations, divalent cations, and combinations thereof.

Active site and/or allosteric inhibitors may inhibit the sulfur producing enzyme from producing sulfur containing species via a competitive, noncompetitive or uncompetitive mechanism. Competitive inhibitors compete with the enzyme's substrate for binding; the competitive inhibitor may bind to the active site or an allosteric site as long as the sulfur containing enzyme is not bound to the substrate. Noncompetitive inhibitors may bind to the sulfur producing enzyme regardless of whether a substrate is bound to the same enzyme; the noncompetitive inhibitor decreases the enzyme's ability to produce sulfur containing compounds. An uncompetitive inhibitor binds to the enzyme-substrate complex, i.e. the sulfur producing enzyme and its substrate complex in a non-limiting example.

The enzyme destabilizer(s) may be added to the base fluid in a concentration that is substantially equal to or more than the amount of sulfur enzyme(s) present in the base fluid and/or the subterranean reservoir wellbore. Alternatively, the concentration of the enzyme destabilizer(s) within the downhole fluid may range from about 1 nM independently to about 1 mM as compared to the total base fluid, alternatively from about 10 nM independently to about 500 nM, or from about 100 nM independently to about 250 nM in another non-limiting embodiment. As used herein with respect to a range, “independently” means that any threshold may be used together with another threshold to give a suitable alternative range.

In another non-limiting embodiment, the base fluid may also include an additional component, such as but not limited to, pH altering/buffering agent to change the pH of the base fluid past that of the enzyme's operational pH range, salts (e.g. KCl and NaCl) for altering the electrostatic stability of the enzyme, and combinations thereof. An amount of the additional component(s) may range from about 1 nM independently to about 5 M, or from about 10 nM independently to about 2 M in another non-limiting embodiment.

In a non-limiting embodiment, the enzyme destabilizer may destabilize at least one sulfur metabolizing enzyme, such as but not limited to ATP: Sulfate Adenylyltransferase (EC 2.7.7.4), Adenoside-5′-phosphosulfonate reductase (EC 1.8.99.2), Sulfite Reductase (EC 1.8.99.1), Sulfite:Oxygen oxidoreductase (EC 1.8.3.1), Hydrogensulfite Reductase (EC 1.8.99.3), Sulfite Reductase (EC 1.8.7.1), O-phosphoserine sulfhydrolase (EC 2.5.1.65), Cysteine Synthase (EC 2.5.1.47), and combinations thereof. In an alternative non-limiting embodiment, the enzyme destabilizer(s) and the additional component(s) may synergistically destabilize the sulfur producing enzyme(s).

In a non-limiting embodiment, the sulfur producing enzyme may maintain optimal function at a temperature ranging from about 75° F. independently to about 180° F., alternatively from about 100° F. independently to about 160° F. The sulfur producing enzyme may maintain optimal function at a pressure less than about 15,000 pounds per square inch (psi). The sulfur producing enzyme may maintain optimal function at a pH ranging from about 4 independently to about 11, alternatively from about 5 independently to about 8. The sulfur producing enzyme may still function at a decreased reaction rate, if at all, outside of the ranges mentioned for temperature, pressure, and/or pH. Thus, the enzyme destabilizer(s) may function within the same or similar ranges for the temperature and/or the pH of the sulfur producing enzyme.

In a non-limiting embodiment, at least one sulfur-species bacteria may encompass the sulfur producing enzyme(s). Sulfur species bacteria is defined as any bacteria capable of producing a sulfur containing compound and/or having at least one sulfur producing enzyme. By destabilizing the sulfur-producing enzyme(s) of the sulfur species bacteria, the enzyme destabilizer(s) and optional additional component(s) may also destabilize the bacteria to the point of decreasing and/or stopping the growth rate of the sulfur-species bacteria. Alternatively, the enzyme destabilizer(s) and optional additional component(s) may have a bactericidal effect on the sulfur species bacteria, i.e. causing the sulfur-species bacteria to die. Therefore, the enzyme destabilizer and optional additional component(s) may decrease and/or inhibit at least one sulfur species bacteria from producing at least one sulfur containing compound, such as but not limited to hydrogen sulfide, mercaptans, thiols, polysulfide, sulfide, cysteine, thioethers, elemental sulfur, and combinations thereof.

In a non-limiting embodiment, the sulfur-species bacteria may be or include any bacteria having at least one sulfur producing enzyme. Non-limiting examples of the sulfur-species bacteria may be or include, but is not limited to, Desulfovibrio vulgaris, Desulfovibrio desulfuricans, Desulfovibrio aespoeensis, Thermodesulfobium narugense, Desulfotomaculum carboxydivorans, Desulfotomaculum ruminis, Desulfovibrio africanus, Desulfovibrio hydrothermalis, Desulfovibrio piezophilus, Desulfobacterium corrodens, Sulfate-reducing bacterium QLNR1, Desulfobacterium catecholicum, Desulfobacterium catecholicum, Desulfobulbus marinus, Desulfobulbus, Desulfobulbus propionicus, Desulfocapsa thiozymogenes, Desulfocapsa sulfexigens, Desulforhopalus vacuolatus, Desulforhopalus, Desulfofustis glycolicus strain, Desulforhopalus singaporensis, Desulfobacterium, Desulfobacterium zeppelinii strain, Desulfobacterium autotrophicum, Desulfobacula phenolica, Desulfobacula toluolica Tol2, Sulfate-reducing bacterium JHA1, Desulfospira joergensenii, Desulfobacter, Desulfobacter postgatei, Desulfotignum, Desulfotignum balticum, Desulforegula conservatrix, Desulfocella, Desulfobotulus sapovorans, Desulfofrigus, Desulfonema magnum, Desulfonema limicola, Desulfobacterium indolicum, Desulfosarcina variabilis, Desulfatibacillum, Desulfococcus multivorans, Desulfococcus, Desulfonema ishimotonii, Desulfococcus oleovorans Hxd3, Desulfococcus niacini, Desulfotomaculum, Desulfotomaculum nigrificans, Desulfotomaculum ruminis, Desulfotomaculum halophilum, Desulfotomaculum acetoxidans, Desulfotomaculum gibsoniae, Desulfotomaculum sapomandens strain, Desulfotomaculum thermosapovorans, Desulfotomaculum, Desulfotomaculum geothermicum, Desulfotomaculum, Desulfosporosinus meridiei, Delta proteobacterium, Thermodesulforhabdus norvegica, Desulfacinum infernum, Desulfacinum hydrothermale, Desulforhabdus amnigena, Desulforhabdus, Desulforhabdus, Desulfomonile tiedjei, Desulfarculus baarsii, Sulfate-reducing bacterium, Sulfate-reducing bacterium, Sulfate-reducing bacterium, Desulfobacterium anilini, Delta proteobacterium, Desulfovibrio profundus strain, Desulfomicrobium baculatum, Desulfocaldus hobo, Desulfovibrio, Desulfovibrio piger, Desulfovibrio ferrophilus, Desulfonatronovibrio hydrogenovorans, Desulfovibrio, Desulfovibrio acrylicus, Desulfovibrio salexigens, Desulfovibrio oxyclinae, Desulfonauticus submarinus, Desulfothermus naphthae, Thermodesulfobacterium, Thermodesulfobacterium hveragerdense, Thermodesulfobacterium thermophilum, Thermodesulfatator indicus, Thermodesulfovibrio yellowstonii, Desulfosporosinus orientis, Desulfotomaculum thermobenzoicum, Desulfotomaculum, Desulfotomaculum, Desulfotomaculum solfataricum, Desulfotomaculum luciae strain, Desulfobacca acetoxidans, Desulfovibrio vulgaris, Desulfovibrio desulfuricans, Desulfovibrio alaskensis, Desulfovibrio magneticus, Desulfosporosinus acidiphilus, Desulfotomaculum kuznetsovii, Desulfotomaculum kuznetsovii, Desulfovibrio sulfodismutans, Desulfomicrobium baculatum, Desulfonatronum lacustre, Desulfohalobium retbaense, Desulfonauticus autotrophicus, Thermodesulfobacterium commune, Thermodesulfobacterium hveragerdense, Thermodesulfovibrio islandicus, Thermodesulfovibrio, Thermodesulfobacterium, Desulfotomaculum thermobenzoicum, Desulfotomaculum thermoacetoxidans, Desulfotomaculum thermocistemum, Desulfotomaculum australicum, Desulfotomaculum kuznetsovii, Desulfovibrio desulfuricans, Desulfovibrio alaskensis, Desulfovibrio vulgaris, Desulfovibrio salexigens, Desulfosporosinus acidiphilus, Desulfosporosinus meridiei, Desulfosporosinus orientis, Desulfotomaculum reducens, and combinations thereof.

In another non-limiting embodiment, the fluid composition having at least one enzyme destabilizer in an effective concentration to decrease an amount of sulfur-containing compounds may be circulated into a subterranean reservoir wellbore. Circulating the fluid composition into the wellbore may decrease an amount of the sulfur-containing compounds in the subterranean reservoir wellbore and/or downhole fluids recovered therefrom. ‘Effective concentration’ is defined herein to mean any concentration of enzyme destabilizer(s) that may decrease or reduce the amount of sulfur-containing compounds within the fluid composition, a subterranean reservoir wellbore and a downhole fluid recovered therefrom; alternatively, ‘effective concentration’ is defined herein to mean any amount of the enzyme destabilizer(s) that may decrease the amount of sulfur-containing compounds.

Parameters that may be used to assess the effectiveness of the enzyme destabilizers may include measurements of the kinetics for the sulfur producing enzymes, amount of sulfur-containing compounds present in the recovered downhole fluids and/or subterranean reservoir wellbore before and after treatment with the enzyme destabilizer(s) and/or fluid composition, and the like. Methods for measuring these parameters may be used to assess the ability of the enzyme destabilizer(s) to reduce, decrease, or inactivate sulfur-containing compounds.

The invention will be further described with respect to the following Examples, which are not meant to limit the invention, but rather to further illustrate the various embodiments.

### EXAMPLES

### Example 1

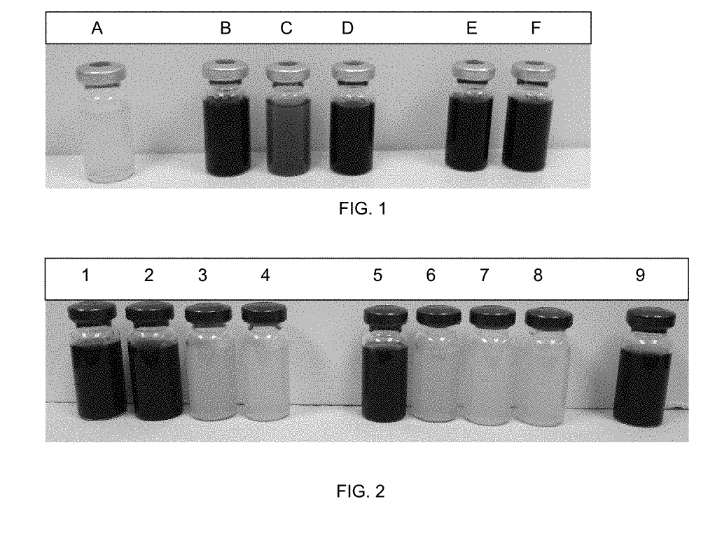

Now turning to the Figures, FIG. 1 illustrates the ability of sterile solutions to produce hydrogen sulfide (H₂S). Six samples were formed with the following procedure. Each sample includes 100 mL of lysogeny broth (LB) (10 mg/L Tryptone, 5 mg/ml yeast extract, 5 mg/mL NaCl, pH 7.5). Sample A is the blank sample, i.e. no sulfur metabolizing enzymes were added. Samples B-D include 0.5 mL of cultured sulfate-reducing bacteria (SRBs) from produced water sources. Samples B, C, and D each include 0.5 mL of cultured sulfate reducing bacteria from produced water sources.

Sample E includes 0.5 mL of supernatant after the same cultured SRBs were additionally sonicated. A QSonic sonicator sonicated the cultured SRBs for one minute cycles at 70% amplitude. The cell debris was removed via centrifugation at 3000 RPM for 25 minutes. 0.5 mL of the remaining supernatant was added to Sample E.

The remaining supernatant was filtered through a 0.2 μM pore size PVDF membrane. 0.5 mL of the filtered supernatant was added to Sample F. No growth was observed after the filtered supernatant was plated on LB-agar plates and incubated for 24 hours at 37° C.; a lack of growth indicated the filtered supernatant was sterile.

All of the samples A-F were then incubated for two days at 37° C.

The presence of hydrogen sulfide (H₂S) is indicated in the color of the sample; a clear color indicates there is no H₂S present in the sample, such as in the blank sample A. A darker color indicates a presence of H₂S in the sample. Here, samples B-F all indicate a presence of H₂S as these samples all have a dark color (i.e. a color that is not clear).

Samples B-D were expected to have a presence of H₂S because the cultured SRBs were added directly to samples B-D. The bacteria was sonicated in Sample E, which effectively opened the bacteria for release of enzymes into the supernatant added to Sample E. The filtration step for the supernatant added to Sample F produced a sterile sample (i.e. no living bacteria present) but allowed the enzymes to pass through the PVDF filter membrane. The dark color of Sample F further indicates that only the enzymes from the SRBs are necessary to produce H₂S, even in the absence of viable SRBs. Therefore, to decrease the amount of sulfur containing components in a given environment, the function of the enzymes from producing sulfur containing compounds must be decreased or inhibited.

### Example 2

FIG. 2 indicates the treatment of samples comprising sulfur producing enzymes after treatment with inhibitors.

Samples 1-9 included the same components as that of Sample F from Example 1, i.e. 0.5 mL of the sterile, enzyme-containing, supernatant. Inhibitors were then added to each of samples 1-9. Samples 1-9 were incubated at 37° C. and monitored periodically. The images of samples 1-9 were taken after 3 months of treatment with an inhibitor and incubated at 37° C.

Sample 1 included 400 ppm iodoacetamide.

Sample 2 included 2 ppt iodoacetamide.

Sample 3 included 5 ppt iodoacetamide.

Sample 4 included 8 ppt iodoacetamide.

Sample 5 included 600 ppm sodium dodecyl sulfate (SDS).

Sample 6 included 3 ppt SDS.

Sample 7 included 7.5 ppt SDS.

Sample 8 included 12 ppt SDS.

Sample 9 was the blank and did not have any inhibitor added thereto.

Similar to Example 1, a clearer color of a sample indicates a sample having less H2S produced than a sample having a darker color by comparison. Here, the inhibitors and the respective amounts of each inhibitor added to sample 3-4 and 6-8 were more effective than the inhibitors and the respective amounts of each inhibitor added to samples 1-2, 5, and 9. This is indicated by the clearer color of samples 3-4 and 6-8 as compared to samples 1-2, 5, and 9.

Iodoacetamide is an active site, enzyme specific inhibitor, while SDS is a general purpose denaturant. Both types of inhibitors were effective in permanently inactivating enzymes. It should also be noted that the enzymes were produced at extremely high concentrations in conditions favorable for bacterial cell growth and enzyme production. Therefore, the concentrations of enzymes used in this experiment is most likely several orders of magnitude larger than what would be experienced in produced water or in reservoir conditions. Much less inhibitor would likely be effective in targeting the same type of enzymes in produced water or in reservoir conditions.

In the foregoing specification, the invention has been described with reference to specific embodiments thereof, and has been described as effective in providing fluid compositions and methods for decreasing and/or removing sulfur containing compounds in fluid compositions circulated in a subterranean reservoir wellbore. However, it will be evident that various modifications and changes can be made thereto without departing from the broader spirit or scope of the invention as set forth in the appended claims. Accordingly, the specification is to be regarded in an illustrative rather than a restrictive sense. For example, specific base fluids, additional components, and the like falling within the claimed parameters, but not specifically identified or tried in a particular composition or method, are expected to be within the scope of this invention.

The present invention may suitably comprise, consist or consist essentially of the elements disclosed and may be practiced in the absence of an element not disclosed. For instance, the fluid composition may consist of or consist essentially of a base fluid selected from the group consisting of a drilling fluid, a completion fluid, a production fluid, a servicing fluid, an injection fluid, a refinery fluid, and combinations thereof; at least one sulfur metabolizing enzyme; at least one enzyme destabilizer selected from the group consisting of a denaturant, an active site inhibitor, an allosteric inhibitor, and combinations thereof; and where the fluid composition comprises a decreased amount of sulfur-containing compounds in the presence of the at least one enzyme destabilizer as compared to an otherwise identical fluid absent the at least one enzyme destabilizer.

The method may consist of or consist essentially of circulating a fluid composition into a subterranean reservoir wellbore and decreasing an amount of the sulfur-containing compounds produced with the at least one sulfur producing enzyme in the subterranean reservoir wellbore and/or downhole fluids recovered therefrom as compared to an identical fluid composition absent the at least one enzyme destabilizer; wherein the fluid composition comprises at least one enzyme destabilizer in an effective concentration to decrease an amount of at least one sulfur producing enzyme; wherein the at least one enzyme destabilizer selected from the group consisting of a denaturant, an active site inhibitor, an allosteric inhibitor, and combinations thereof.

The words “comprising” and “comprises” as used throughout the claims, are to be interpreted to mean “including but not limited to” and “includes but not limited to”, respectively.

What is claimed is:

1. A fluid composition comprising:
a base fluid selected from the group consisting of drilling fluids, servicing fluids, production fluids, completion fluids, injection fluids, refinery fluids, and combinations thereof; and
at least one sulfur producing enzyme;
at least one enzyme destabilizer selected from the group consisting of a denaturant, an active site inhibitor, an allosteric inhibitor, and combinations thereof;
wherein the fluid composition comprises a decreased amount of sulfur-containing compounds in the presence of the at least one enzyme destabilizer as compared to an otherwise identical fluid absent the at least one enzyme destabilizer.

2. The fluid composition of claim 1, wherein the concentration of the at least one enzyme destabilizer within the fluid composition ranges from about 1 nM to about 1 mM as compared to the total base fluid.

3. The fluid composition of claim 1, wherein the fluid composition further comprises an additional component selected from the group consisting of pH altering agents, pH buffering agents, salts for altering the electrostatic stability of the enzyme, and combinations thereof.

4. The fluid composition of claim 1, wherein the at least one sulfur producing enzyme is selected from the group consisting of ATP: sulfate adenylyltransferase, adenoside-5′-phosphosulfonate reductase, sulfite reductase, sulfite:oxygen oxidoreductase, hydrogensulfite reductase, sulfite reductase, O-phosphoserine sulfhydrolase, cysteine synthase, and combinations thereof.

5. The fluid composition of claim 1, wherein at least one sulfur-species bacteria comprises the at least one sulfur producing enzyme; wherein the at least one sulfur-species bacteria is selected from the group consisting of Desulfovibrio vulgaris, Desulfovibrio desulfuricans, Desulfovibrio aespoeensis, Thermodesulfobium narugense, Desulfotomaculum carboxydivorans, Desulfotomaculum ruminis, Desulfovibrio africanus, Desulfovibrio hydrothermalis, Desulfovibrio piezophilus, Desulfobacterium corrodens, Sulfate-reducing bacterium QLNR1, Desulfobacterium catecholicum, Desulfobacterium catecholicum, Desulfobulbus marinus, Desulfobulbus, Desulfobulbus propionicus, Desulfocapsa thiozymogenes, Desulfocapsa sulfexigens, Desulforhopalus vacuolatus, Desulforhopalus, Desulfofustis glycolicus strain, Desulforhopalus singaporensis, Desulfobacterium, Desulfobacterium zeppelinii strain, Desulfobacterium autotrophicum, Desulfobacula phenolica, Desulfobacula toluolica Tol2, Sulfate-reducing bacterium JHA1, Desulfospira joergensenii, Desulfobacter, Desulfobacter postgatei, Desulfotignum, Desulfotignum balticum, Desulforegula conservatrix, Desulfocella, Desulfobotulus sapovorans, Desulfofrigus, Desulfonema magnum, Desulfonema limicola, Desulfobacterium indolicum, Desulfosarcina variabilis, Desulfatibacillum, Desulfococcus multivorans, Desulfococcus, Desulfonema ishimotonii Desulfococcus oleovorans Hxd3, Desulfococcus niacini, Desulfotomaculum, Desulfotomaculum nigrificans, Desulfotomaculum ruminis, Desulfotomaculum halophilum, Desulfotomaculum acetoxidans, Desulfotomaculum gibsoniae, Desulfotomaculum sapomandens strain, Desulfotomaculum thermosapovorans, Desulfotomaculum, Desulfotomaculum geothermicum, Desulfotomaculum, Desulfosporosinus meridiei, Delta proteobacterium, Thermodesulforhabdus norvegica, Desulfacinum infernum, Desulfacinum hydrothermale, Desulforhabdus amnigena, Desulforhabdus, Desulforhabdus, Desulfomonile tiedjei, Desulfarculus baarsii, Sulfate-reducing bacterium, Sulfate-reducing bacterium, Sulfate-reducing bacterium, Desulfobacterium anilini, Delta proteobacterium, Desulfovibrio profundus strain, Desulfomicrobium baculatum, Desulfocaldus hobo, Desulfovibrio, Desulfovibrio piger, Desulfovibrio ferrophilus, Desulfonatronovibrio hydrogenovorans, Desulfovibrio, Desulfovibrio acrylicus, Desulfovibrio salexigens, Desulfovibrio oxyclinae, Desulfonauticus submarinus, Desulfothermus naphthae, Thermodesulfobacterium, Thermodesulfobacterium hveragerdense, Thermodesulfobacterium thermophilum, Thermodesulfatator indicus, Thermodesulfovibrio yellowstonii, Desulfosporosinus orientis, Desulfotomaculum thermobenzoicum, Desulfotomaculum, Desulfotomaculum, Desulfotomaculum solfataricum, Desulfotomaculum luciae strain, Desulfobacca acetoxidans, Desulfovibrio vulgaris, Desulfovibrio desulfuricans, Desulfovibrio alaskensis, Desulfovibrio magneticus, Desulfosporosinus acidiphilus, Desulfotomaculum kuznetsovii, Desulfotomaculum kuznetsovii, Desulfovibrio sulfodismutans, Desulfomicrobium baculatum, Desulfonatronum lacustre, Desulfohalobium retbaense, Desulfonauticus autotrophicus, Thermodesulfobacterium commune, Thermodesulfobacterium hveragerdense, Thermodesulfovibrio islandicus, Thermodesulfovibrio, Thermodesulfobacterium, Desulfotomaculum thermobenzoicum, Desulfotomaculum thermoacetoxidans, Desulfotomaculum thermocisternum, Desulfotomaculum australicum, Desulfotomaculum kuznetsovii, Desulfovibrio desulfuricans, Desulfovibrio alaskensis, Desulfovibrio vulgaris, Desulfovibrio salexigens, Desulfosporosinus acidiphilus, Desulfosporosinus meridiei, Desulfosporosinus orientis, Desulfotomaculum reducens, and combinations thereof.

6. The fluid composition of claim 1, wherein the at least one enzyme destabilizer is selected from the group consisting of malimide, sodium flurophosphate, iodoacetamide, sodium dodecyl sulfate, sodium chromium tetrahydrate, 5,5′-dithiobis(2-nitrobenzoic acid), sodium molybdate, and combinations thereof.

7. The fluid composition of claim 1, wherein the at least one sulfur containing compound is selected from the group consisting of hydrogen sulfide, mercaptans, thiols, polysulfide, sulfide, cysteine, thioethers, elemental sulfur, and combinations thereof.

8. A method comprising:
circulating a fluid composition into a subterranean reservoir wellbore; wherein the fluid composition comprises at least one enzyme destabilizer in an effective concentration to decrease an amount of at least one sulfur producing enzyme; wherein the at least one enzyme destabilizer selected from the group consisting of a denaturant, an active site inhibitor, an allosteric inhibitor, and combinations thereof; and
decreasing an amount of the sulfur-containing compounds produced with the at least one sulfur producing enzyme in the subterranean reservoir wellbore and/or downhole fluids recovered therefrom as compared to an identical fluid composition absent the at least one enzyme destabilizer.

9. The method of claim 8, wherein the concentration of the at least one enzyme destabilizer within the fluid composition ranges from about 1 nM to about 1 mM as compared to the total fluid composition.

10. The method of claim 8, wherein the fluid composition further comprises an additional component selected from the group consisting of pH altering agents, pH buffering agents, salts for altering the electrostatic stability of the enzyme, and combinations thereof.

11. The method of claim 8, wherein the at least one sulfur producing enzyme is selected from the group consisting of ATP: sulfate adenylyltransferase, adenoside-5′-phosphosulfonate reductase, sulfite reductase, sulfite:oxygen oxidoreductase, hydrogensulfite reductase, sulfite reductase, O-phosphoserine sulfhydrolase, cysteine synthase, and combinations thereof.

12. The method of claim 8, wherein at least one sulfur-species bacteria comprises the at least one sulfur producing enzyme; wherein the at least one sulfur-species bacteria is selected from the group consisting of Desulfovibrio vulgaris, Desulfovibrio desulfuricans, Desulfovibrio aespoeensis, Thermodesulfobium narugense, Desulfotomaculum carboxydivorans, Desulfotomaculum ruminis, Desulfovibrio africanus, Desulfovibrio hydrothermalis, Desulfovibrio piezophilus, Desulfobacterium corrodens, Sulfate-reducing bacterium QLNR1, Desulfobacterium catecholicum, Desulfobacterium catecholicum, Desulfobulbus marinus, Desulfobulbus, Desulfobulbus propionicus, Desulfocapsa thiozymogenes, Desulfocapsa sulfexigens, Desulforhopalus vacuolatus, Desulforhopalus, Desulfofustis glycolicus strain, Desulforhopalus singaporensis, Desulfobacterium, Desulfobacterium zeppelinii strain, Desulfobacterium autotrophicum, Desulfobacula phenolica, Desulfobacula toluolica Tol2, Sulfate-reducing bacterium JHA1, Desulfospira joergensenii, Desulfobacter, Desulfobacter postgatei, Desulfotignum, Desulfotignum balticum, Desulforegula conservatrix, Desulfocella, Desulfobotulus sapovorans, Desulfofrigus, Desulfonema magnum, Desulfonema limicola, Desulfobacterium indolicum, Desulfosarcina variabilis, Desulfatibacillum, Desulfococcus multivorans, Desulfococcus, Desulfonema ishimotonii Desulfococcus oleovorans Hxd3, Desulfococcus niacini, Desulfotomaculum, Desulfotomaculum nigrificans, Desulfotomaculum ruminis, Desulfotomaculum halophilum, Desulfotomaculum acetoxidans, Desulfotomaculum gibsoniae, Desulfotomaculum sapomandens strain, Desulfotomaculum thermosapovorans, Desulfotomaculum, Desulfotomaculum geothermicum, Desulfotomaculum, Desulfosporosinus meridiei, Delta proteobacterium, Thermodesulforhabdus norvegica, Desulfacinum infernum, Desulfacinum hydrothermale, Desulforhabdus amnigena, Desulforhabdus, Desulforhabdus, Desulfomonile tiedjei, Desulfarculus baarsii, Sulfate-reducing bacterium, Sulfate-reducing bacterium, Sulfate-reducing bacterium, Desulfobacterium anilini, Delta proteobacterium, Desulfovibrio profundus strain, Desulfomicrobium baculatum, Desulfocaldus hobo, Desulfovibrio, Desulfovibrio piger, Desulfovibrio ferrophilus, Desulfonatronovibrio hydrogenovorans, Desulfovibrio, Desulfovibrio acrylicus, Desulfovibrio salexigens, Desulfovibrio oxyclinae, Desulfonauticus submarinus, Desulfothermus naphthae, Thermodesulfobacterium, Thermodesulfobacterium hveragerdense, Thermodesulfobacterium thermophilum, Thermodesulfatator indicus, Thermodesulfovibrio yellowstonii, Desulfosporosinus orientis, Desulfotomaculum thermobenzoicum, Desulfotomaculum, Desulfotomaculum, Desulfotomaculum solfataricum, Desulfotomaculum luciae strain, Desulfobacca acetoxidans, Desulfovibrio vulgaris, Desulfovibrio desulfuricans, Desulfovibrio alaskensis, Desulfovibrio magneticus, Desulfosporosinus acidiphilus, Desulfotomaculum kuznetsovii, Desulfotomaculum kuznetsovii, Desulfovibrio sulfodismutans, Desulfomicrobium baculatum, Desulfonatronum lacustre, Desulfohalobium retbaense, Desulfonauticus autotrophicus, Thermodesulfobacterium commune, Thermodesulfobacterium hveragerdense, Thermodesulfovibrio islandicus, Thermodesulfovibrio, Thermodesulfobacterium, Desulfotomaculum thermobenzoicum, Desulfotomaculum thermoacetoxidans, Desulfotomaculum thermocisternum, Desulfotomaculum australicum, Desulfotomaculum kuznetsovii, Desulfovibrio desulfuricans, Desulfovibrio alaskensis, Desulfovibrio vulgaris, Desulfovibrio salexigens, Desulfosporosinus acidiphilus, Desulfosporosinus meridiei, Desulfosporosinus orientis, Desulfotomaculum reducens, and combinations thereof.

13. The method of claim 8, wherein the at least one enzyme destabilizer is selected from the group consisting of malimide, sodium flurophosphate, iodoacetamide, sodium dodecyl sulfate, sodium chromium tetrahydrate, 5,5′-dithiobis(2-nitrobenzoic acid), sodium molybdate, and combinations thereof.

14. The method of claim 8, wherein the at least one sulfur containing compound is selected from the group consisting of hydrogen sulfide, mercaptans, thiols, polysulfide, sulfide, cysteine, thioethers, elemental sulfur, and combinations thereof.

15. The method of claim 8, wherein the fluid composition comprises a base fluid selected from the group consisting of drilling fluids, servicing fluids, production fluids, completion fluids, injection fluids, refinery fluids, and combinations thereof.
